(19) United States
(12) Patent Application Publication
(10) Pub. No.: US 2020/0380435 A1
(43) Pub. Date: Dec. 3, 2020

(54) SYSTEM AND METHOD TO CHARACTERIZE RISK OF OCCUPATIONAL HEALTH HAZARDS AND IMPLEMENT CONTROL MEASURES TO PROTECT WORKERS, WITH REAL TIME EVALUATIONS OF ENVIRONMENTAL CONDITIONS AND EXPOSURE CONCENTRATIONS

(71) Applicant: Jeffrey B. Miller, New Athens, IL (US)

(72) Inventor: Jeffrey B. Miller, New Athens, IL (US)

(73) Assignee: IAM Creative Concepts, LLC, New Athens, IL (US)

(21) Appl. No.: 16/885,064

(22) Filed: May 27, 2020

Related U.S. Application Data
(60) Provisional application No. 62/854,780, filed on May 30, 2019.

(51) Int. Cl. G06Q 10/06; G06Q 50/26; G06Q 30/00; G06Q 10/10; G16H 50/30; G06F 16/22; G06F 16/21; G06N 5/02; G06N 20/00

(52) U.S. Cl. CPC G06Q 10/0635; G06Q 50/265; G06Q 30/018; G06Q 10/105; G16H 10/60; G06F 16/22; G06F 16/21; G06N 5/02; G06N 20/00; G16H 50/30

(57) ABSTRACT

A system and method to characterize risk of occupational health hazards and implement control measures to protect workers, with real time evaluations of environmental conditions and exposure concentrations, and platform for use are disclosed herein. The present disclosure provides a system and method for characterizing the risk of various occupational health hazards across various industries, implementing and providing control measures that can be adopted to protect workers in such hazards or situations, and even provide real-time evaluations and recommendations based on existing conditions of environmental conditions to mitigate exposure concentrations of various hazards that may be experienced by or induced upon a worker in an environment.

### CROSS-REFERENCE TO RELATED APPLICATIONS

The present application claims priority to U.S. Provisional Patent Application No. 62/854,780 which was filed on May 30, 2019, which is hereby incorporated by reference herein in its entirety, including any figures, tables, or drawings.

### FIELD OF THE DISCLOSURE

The present disclosure relates generally to occupational health. More specifically, the present disclosure is a system and method for the characterization of risks associated with various types of occupational health hazards and a system and method for implementing control measures to protect workers from identified occupational health hazards. Additionally, and without limitation, the present disclosure provides real time evaluations of occupational health hazards and environmental conditions, including but not limited to, exposure concentrations of health hazards.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document: Copyright Jeffrey Miller. All rights reserved.

### BACKGROUND OF THE DISCLOSURE

Environmental, Health and Safety (EHS) industry is well known in the art. Generally, Environmental Health and Safety is the implementation of practices to manage work environments and make the workplace and/or work environment safer. Said another way, the implementation of health and safety practices for a workplace are for the purpose of making sure work activities do not cause harm to anyone.

Organized efforts, from a top down approach are often necessary to create a safe environment. Thus, the safety portion of environmental health and safety comes into play. From the safety standpoint, an organization creates practices and procedures for various types of work environments which prevent and/or reduce the risk of harm that can come to the workers, and others, in a particular environment.

Not only is the health and safety of workers at play, but environmental aspects as well. From an environmental perspective, many regulations exist which are for the health and improvement of the environment in which we all live. These regulations, and the environmental protection act as one example, involve limiting quantities of pollutants and the like for limiting the disruption of environmental function and destruction. Environmental health and safety includes a systematic approach to abiding by various regulations, managing waste emissions, managing pollutant emissions, managing contaminant introduction, and the like for various environments.

Environmental health and safety, from a human perspective, generally applies to those humans within an environment; in other words those with exposure to an environment or environmental factors. An industrial hygienist is a person who manages these environments. In other words, an industrial hygienist is an individual or group of people involved in analyzing, identifying, and measuring workplace hazards and stressors that may cause sickness, impaired health, or discomfort, being physical discomfort and or psychosocial discomfort caused among and/or to workers within an environment in which a work is to perform their duties. Some of the various discomforts which may be analyzed by an industrial hygienist include, but are not limited to, chemical analysis, chemical identification, chemical measuring, analyzing the physical environment and factors such as noise and radiation and more, as well as ergonomic and biological exposures.

Workers may be exposed to a variety of factors, such as chemical exposure, biological exposure, ergonomic stresses, and a combination of these and other stressors. A worker in any environment is a person performing a task necessary for operations to be carried out. Whether these operations are for a company or an individual, many such operations require exposure to varying degrees of stressors. These varying workplaces with varying levels of exposure are where any worker or plurality of worker will be situated. This environment, the workplace, is the environment that is important to an industrial hygienist, also known as an occupational hygienist.

Chemical Substances are one important factor measured by an industrial hygienist to determine the overall stress level of a workplace. A chemical substance, or chemical stressor, is generally a hazardous substance, which when present in an environment, can cause damage to living organisms within the environment. Alternatively, the presence of a chemical substance, which when present in an environment, causes an ecosystem to be disrupted or destroyed is also considered a chemical stressor. Even the slight presence of a chemical substance, a level which reduces full functionality of an organism or environment is considered a stressor.

Health Hazards are more predominantly used to describe stressors within an environment which cause the health of an organism to lessen in any manner. Some examples of common health hazard risks which are associated with chemical substances and/or environmental stressors are breathing problems, skin irritations, reduced wellbeing, hearing damage, or more severely, bone and/or muscle damage, or even death.

Biological Agents present one such stressor and range from minor stressors to severe stressors. Some examples of biological stressors include, but are not limited to, viruses, bacteria, insects, animals and or other biological stressors that cause or can present adverse health impacts. Some additional examples that can cause adverse health impacts, and which are measurable in an environment are the presence of mould, blood or other bodily fluids, harmful plants which may present as an allergen, sewage, dust, a combination of these agents, and the like.

Ergonomic Stressors include naturally occurring environmental ergonomic stressors as well as equipment induced ergonomic stressors and, additionally, other physical stressors which result from exposure to an environment over a duration of time. Many jobs task workers with tasks and physical stress from equipment and/or from performance which result in stress, strain, or overexertion which can be harmful, especially over a prolonged period and/or prolonged exposure of time. Some examples of a stressor over a period of time include uncomfortable position of the body, or forceful exertions made by the body. These ergonomic stressors can be common and are measurable.

While these types of stressors have been common in nearly every task performed by mankind for all of history, these various stressors have been hard to measure, hard to analyze, hard to calculate, and hard to make sense out of. In situations where multiple stressors, and multiple types of stressors exist in a single environment, calculating the effects of these stressors alone, or in combination has been nearly impossible. Needless to say, any type of instantaneous calculation of the effects of a plurality of these stressors is long felt, but never has been considered achievable in the state of the art. Furthermore, no calculation which includes a unique individual within an environment, down to genomic make-up has ever been considered due the difficulty.

Additionally, there are only so many industrial hygienists. The specialty requires extensive education and time. Thus, hiring industrial hygienists and employing industrial hygienists can be costly and not easy for all entities to attain. Furthermore, even when large amounts of resources are available for industrial hygienists, the number of work environments, combined with the number of unique individuals which may be present in those environments creates a great number of calculations, analysis, and the like which need to be performed by an industrial hygienist. Even if there were ways for a industrial hygienist to make these calculations accurately and effectively, there wouldn't be enough time given the number of environments globally, the number of works, which is in the billions, and the number of industrial hygienists available. Thus, there is a great need in the art for an efficient and effective way of providing these services to make the services available for a greater number of workers and workplaces.

Thus, there exists a need in the art for a safe and effective means to protect employees and identify health hazards efficiently and effectively. Furthermore, there exists a long felt need in the art for a system which can identify health hazards one may be exposed to during any given task involved in a particular job. Furthermore, there exists a need in the art for a system which can identify these health hazards and how those health hazards will affect a unique individual. Many jobs and/or tasks have a plurality of health hazards associated with the job and/or task. So there exists a need in the art for a system and/or mechanism which can handle the computation of a plurality of health hazards and the combination of these health hazards. It is evident that the art has a long felt need for a system capable of handling these items along with providing a standardized means of performing these tasks and providing reports and/or other measurables.

### SUMMARY OF THE DISCLOSURE

The present disclosure serves employers, employees, and other users in various industries to provide solutions for these problems and more. The present disclosure is a system and method to characterize risk of occupational health hazards and implement control measures to protect workers, with real time evaluations of environmental conditions and exposure concentrations. Said another way, the present disclosure provides a system and method of use for an industrial hygienist to quickly and efficiently evaluate exposure risk assessments in real time as well as provide near instant mediation recommendations for controlling and mitigating exposures.

The present disclosure provides a solution to the problems existing in the art, the problems outlined herein, and many more. The first step of the present disclosure, and the first step of remediation of stressors on a worker in a workplace, is to identify environmental stressors. Depending on the worker, and depending on the environment and/or task the worker is exposed to, the number of hazards could be a few or it could be great. Identifying all of these hazards for the entirety of completion of the workers task is the first, and one of the most important steps, in successfully reducing the health and safety exposure. There is no standardized system for identifying such stressors. Furthermore, only, roughly, 500 chemical stressors are ever identified. This number is far greater when one considers the number of chemical combinations a worker could be exposed to along with biological and ergonomic factors, and the like. Thus, one object of the present disclosure is that the present disclosure provides a standardized system. The standardized system presents standardized tasks with measurables and the like so that there is a standard method.

The second step is to make the standardized system more readily available. The number of qualified individuals who can identify these risks are limited to highly qualified industrial hygienists. Thus, another object of the disclosure is to utilize computer systems, along with cloud computing systems and application programming interfaces, and other interfaces, to make more readily available the present disclosure in a form which can be used and managed by a plurality of individuals, not limited to highly qualified industrial hygienists. In this way, at least the present disclosure can be more readily available, thus more environmental stressors and the like can be identified.

By providing a standardized identification system, the user in any industry can begin to tackle these various exposure risks. In this way, a user can also have readily available to them the present disclosure, which provides for the next steps in the process as well as guides a user through the process. The next step of the process is to calculate the levels of the identified stressors.

The third step provided by the disclosure herein is to calculate the level of the identified stressors. The present disclosure provides an easy means, that even a novice can perform using the interface which will measure the level of the identified stressor.

The fourth step, and provided by the present disclosure includes organizing all the information entered—including stressors but also personally identifying information about the worker and or workers exposed to the environment, which may include genomics and more related to effects a stressor may have on a unique individual—and in addition to organization of the information entered, the present disclosure provides for storage of the information and machine learning such that information about a certain geographical location can be stored and saved for future use and further calculations. As geolocations merge, the present disclosure provides for machine learning which can merge the information, such as provide alerts for chemicals mixing or chemicals being blown by a wind current to another geographical location, and the relative effects. In this way, the present disclosure provides for memory and storage of the various tasks, ergonomic factors of tasks, and exposures and machine learning of this information and integration with similar tasks in new locations and the like.

Thus, it is a major step and major goal of the present disclosure to provide a user with a user friendly system and method of use for guiding a user through a step-by-step task exposure risk assessment (also known as “TERA”), and do this in real time, while providing in real time solution options. Additionally, the present disclosure provides this for unique settings and unique individuals, standardization, combining tasks, combining references, and task conditions in a way where real time recommendations can be provided to reduce and/or remediate stressors. Said another way, the present disclosure, for the first time, provides organizations with an objective, learning database related to environmental health and occupational safety. Furthermore, the present disclosure provides for uploading sampling data of an environment, such as air quality, water quality, and more, for these uploads to be used in the calculations and the like. This data can be leveraged by many companies to reduce the cost of sampling and the like.

Said another way, the present disclosure provides a system and method for combining references, standards, and adjusting task conditions in a single program and/or system, which in turn generates control recommendations. In this way, the system provides a tool and resource. Additionally, the present disclosure removes human subjectivity. Additionally, the present system stores data along with geographical location or unique individual information, as relevant. Additionally,

The present disclosure provides 1) a resource center, 2) a standardization form; 3) a task exposure risk assessment; and 4) an easy to use interface with historical data and machine learning.

Thus, it is a primary object of the disclosure to provide a system and method to characterize risk of occupational health hazards and implement control measures to protect workers, with real time evaluations of environmental conditions and exposure concentrations that improves upon the state of the art.

Another object of the disclosure is to provide a system and method to characterize risk of occupational health hazards and implement control measures to protect workers, with real time evaluations of environmental conditions and exposure concentrations that is easy to use.

Yet another object of the disclosure is to provide a system and method to characterize risk of occupational health hazards and implement control measures to protect workers, with real time evaluations of environmental conditions and exposure concentrations that is high quality.

Another object of the disclosure is to provide a system and method to characterize risk of occupational health hazards and implement control measures to protect workers, with real time evaluations of environmental conditions and exposure concentrations that is user friendly.

Yet another object of the disclosure is to provide a system and method to characterize risk of occupational health hazards and implement control measures to protect workers, with real time evaluations of environmental conditions and exposure concentrations that provides a guide through a task exposure risk assessment.

Another object of the disclosure is to provide a system and method to characterize risk of occupational health hazards and implement control measures to protect workers, with real time evaluations of environmental conditions and exposure concentrations that provides a step-by-step guide for a user on how to reduce exposure of stressors in a work environment and/or workplace.

Yet another object of the disclosure is to provide a system and method to characterize risk of occupational health hazards and implement control measures to protect workers, with real time evaluations of environmental conditions and exposure concentrations that combines references.

Another object of the disclosure is to provide a system and method to characterize risk of occupational health hazards and implement control measures to protect workers, with real time evaluations of environmental conditions and exposure concentrations that combines standards.

Yet another object of the disclosure is to provide a system and method to characterize risk of occupational health hazards and implement control measures to protect workers, with real time evaluations of environmental conditions and exposure concentrations that combines task conditions into a single analysis.

Another object of the disclosure is to provide a system and method to characterize risk of occupational health hazards and implement control measures to protect workers, with real time evaluations of environmental conditions and exposure concentrations that generates control recommendations.

Yet another object of the disclosure is to provide a system and method to characterize risk of occupational health hazards and implement control measures to protect workers, with real time evaluations of environmental conditions and exposure concentrations that provides companies and/or other entities with a tool and resource for task exposure assessment and recommendations.

Another object of the disclosure is to provide a system and method to characterize risk of occupational health hazards and implement control measures to protect workers, with real time evaluations of environmental conditions and exposure concentrations that provides data leveraging.

Yet another object of the disclosure is to provide a system and method to characterize risk of occupational health hazards and implement control measures to protect workers, with real time evaluations of environmental conditions and exposure concentrations that provides a database for environmental conditions to be stored.

Another object of the disclosure is to provide a system and method to characterize risk of occupational health hazards and implement control measures to protect workers, with real time evaluations of environmental conditions and exposure concentrations that provides a database for unique individual information to be stored.

Yet another object of the disclosure is to provide a system and method to characterize risk of occupational health hazards and implement control measures to protect workers, with real time evaluations of environmental conditions and exposure concentrations that provides a means of accessing location information uploads by a plurality of users and/or companies.

Another object of the disclosure is to provide a system and method to characterize risk of occupational health hazards and implement control measures to protect workers, with real time evaluations of environmental conditions and exposure concentrations that removes human subjectivity from the identification.

Yet another object of the disclosure is to provide a system and method to characterize risk of occupational health hazards and implement control measures to protect workers, with real time evaluations of environmental conditions and exposure concentrations that removes human subjectivity from the analysis.

Another object of the disclosure is to provide a system and method to characterize risk of occupational health hazards and implement control measures to protect workers, with real time evaluations of environmental conditions and exposure concentrations that removes human subjectivity from the recommendation phase.

Yet another object of the disclosure is to provide a system and method to characterize risk of occupational health hazards and implement control measures to protect workers, with real time evaluations of environmental conditions and exposure concentrations that combines information from a plurality of references and standards in a user friendly format.

Another object of the disclosure is to provide a system and method to characterize risk of occupational health hazards and implement control measures to protect workers, with real time evaluations of environmental conditions and exposure concentrations that provides for sample upload.

Yet another object of the disclosure is to provide a system and method to characterize risk of occupational health hazards and implement control measures to protect workers, with real time evaluations of environmental conditions and exposure concentrations that provides confidentiality.

Another object of the disclosure is to provide a system and method to characterize risk of occupational health hazards and implement control measures to protect workers, with real time evaluations of environmental conditions and exposure concentrations that provides for unique task exposure risk assessment creation based on geographic location.

Yet another object of the disclosure is to provide a system and method to characterize risk of occupational health hazards and implement control measures to protect workers, with real time evaluations of environmental conditions and exposure concentrations that provides for unique task exposure risk assessment creation based on an individual.

Another object of the disclosure is to provide a system and method to characterize risk of occupational health hazards and implement control measures to protect workers, with real time evaluations of environmental conditions and exposure concentrations that provides unique task exposure risk assessment creation based on geographic location and a unique user or unique group of users.

Yet another object of the disclosure is to provide a system and method to characterize risk of occupational health hazards and implement control measures to protect workers, with real time evaluations of environmental conditions and exposure concentrations that provides sampling data to be confidentially linked to a specific task exposure risk assessment creating an objective database that can be leveraged by other companies, thereby reducing cost of exposure data collection.

Another object of the disclosure is to provide a system and method to characterize risk of occupational health hazards and implement control measures to protect workers, with real time evaluations of environmental conditions and exposure concentrations that provides a resource for environmental health and safety.

Yet another object of the disclosure is to provide a system and method to characterize risk of occupational health hazards and implement control measures to protect workers, with real time evaluations of environmental conditions and exposure concentrations that provides a resource center for environmental health and safety.

Another object of the disclosure is to provide a system and method to characterize risk of occupational health hazards and implement control measures to protect workers, with real time evaluations of environmental conditions and exposure concentrations that provides standardization and standard forms for environmental health and safety.

Yet another object of the disclosure is to provide a system and method to characterize risk of occupational health hazards and implement control measures to protect workers, with real time evaluations of environmental conditions and exposure concentrations that provides a task exposure risk assessment process and method of completing the process to gain exposure recommendations.

Another object of the disclosure is to provide a system and method to characterize risk of occupational health hazards and implement control measures to protect workers, with real time evaluations of environmental conditions and exposure concentrations that provides a user friendly interface and or dashboard for storing, navigating, entering, and accessing information related to environmental health and safety.

Another object of the disclosure is to provide a system and method to characterize risk of occupational health hazards and implement control measures to protect workers, with real time evaluations of environmental conditions and exposure concentrations that is durable.

Yet another object of the disclosure is to provide a system and method to characterize risk of occupational health hazards and implement control measures to protect workers, with real time evaluations of environmental conditions and exposure concentrations that is robust.

Another object of the disclosure is to provide a system and method to characterize risk of occupational health hazards and implement control measures to protect workers, with real time evaluations of environmental conditions and exposure concentrations that can be used from a mobile device.

These and other objects, features, or advantages of the present disclosure will become apparent from the specification and claims.

### BRIEF DESCRIPTION OF THE DRAWINGS

FIG. 1 is a diagram illustrating the various phases and parts of the system; the view showing a first standardized forms component; the view showing showing a second standardized forms component; the view showing a legend; the view showing a resource center; the view showing a task exposure risk assessment builder; the view showing a dashboard;

FIG. 2 is a diagram illustrating a standardized form layout for the second major part of the system; the view showing standardized forms section having data tables; the view showing reference to standardized forms having hazard information;

FIG. 3 is a diagram illustrating a standardized form layout for the third major part of the system; the view showing the resource center layout; the view showing the resource center layout having data tables; the view showing the resource center layout having hazard information; the view showing the resource center layout having references; the view showing the resource center layout having an objective database;

FIG. 4 is a diagram illustrating a task exposure risk assessment builder; the view showing the task exposure risk assessment builder having user details; the view showing the task exposure risk assessment builder having a task classification; the view showing the task exposure risk assessment builder having hazard information; the view showing the task exposure risk assessment builder having work conditions; the view showing the task exposure risk assessment builder having control recommendations; the view showing the task exposure risk assessment builder having a review; the view showing the task exposure risk assessment builder having an output;

FIG. 5 is a diagram illustrating a user dashboard; the view showing the user dashboard having company analytics; the view showing the user dashboard having employee and/or worker information; the view showing the user dashboard having entity data tables; the view showing the user dashboard having entity hazard information; the view showing the user dashboard having an objective database; the view showing the user dashboard having combined task exposure risk assessment information; the view showing the user dashboard having compliance information;

FIG. 6 is a diagram illustrating a legend; the view showing a resource center description; the view showing a standardized forms description; the view showing a task exposure risk assessment builder description; the view showing a dashboard and/or company description;

FIG. 7 is a diagram illustrating the various phases and parts of the system; the view showing a first standardized forms component; the view showing showing a second standardized forms component; the view showing a legend; the view showing a resource center; the view showing a task exposure risk assessment builder; the view showing a dashboard;

FIG. 8 is a diagram illustrating a standardized form layout; the view showing standardized forms section having data tables;

FIG. 9 is a diagram illustrating a standardized form layout; the view showing the resource center layout; the view showing the standardized form layout having hazard information; the view showing a chemical form; the view showing a substance form;

FIG. 10 is a diagram illustrating a resource center; the view showing the resource center having a plurality of data tables; the view showing the resource center having hazard information; the view showing the resource center having references; the view showing the resource center having an objective database;

FIG. 11 is a diagram illustrating a task exposure risk assessment builder; the view showing the task exposure risk assessment builder having user details; the view showing the task exposure risk assessment builder having task classifications; the view showing the task exposure risk assessment builder having hazard information; the view showing the task exposure risk assessment builder having work conditions; the view showing the task exposure risk assessment builder having control recommendations; the view showing the task exposure risk assessment builder having task exposure risk assessment reviews; the view showing the task exposure risk assessment builder having task exposure risk assessment final outputs;

FIG. 12 is a diagram illustrating a company and/or entity and/or user dashboard; the view showing the user dashboard having company analytics; the view showing the user dashboard having employee and/or worker information and/or data; the view showing the user dashboard having data tables; the view showing the user dashboard having hazard information; the view showing the user dashboard having an objective database; the view showing the user dashboard having a combined company task exposure risk assessment;

FIG. 13 is a diagram illustrating a legend; the view showing a description of the resource center; the view showing a description of the standard forms; the view showing a description of the task exposure risk assessment builder; the view showing a description of the dashboard;

FIG. 14 is a flowchart diagram showing the steps of the step-by-step guide for a user engaging with the present disclosure; the view showing a home page; the view showing a chemical task exposure risk assessment; the view showing a user information step; the view showing a chemical selection and/or constituent chemical selection step; the view showing a work conditions step; the view showing task information step; the view showing a task exposure risk assessment summary step;

FIG. 15 is a diagram illustrating user information of the system; the view showing various parameters associated with user information;

FIG. 16 is a diagram illustrating chemical information of a chemical in the system; the view showing various parameters associated with a chemical;

FIG. 17 is a diagram illustrating constituent information; the view showing various constituent information parameters;

FIG. 18 is a diagram illustrating work conditions; the view showing various work condition elements; the view showing various work condition ratings;

FIG. 19 is a diagram illustrating task information; the view showing various parameters associated with task information;

FIG. 20 is a diagram illustrating a review; the view showing various information associated with the entry.

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

All illustrations of the drawings are for the purpose of describing selected versions of the present invention and are not intended to limit the scope of the present invention.

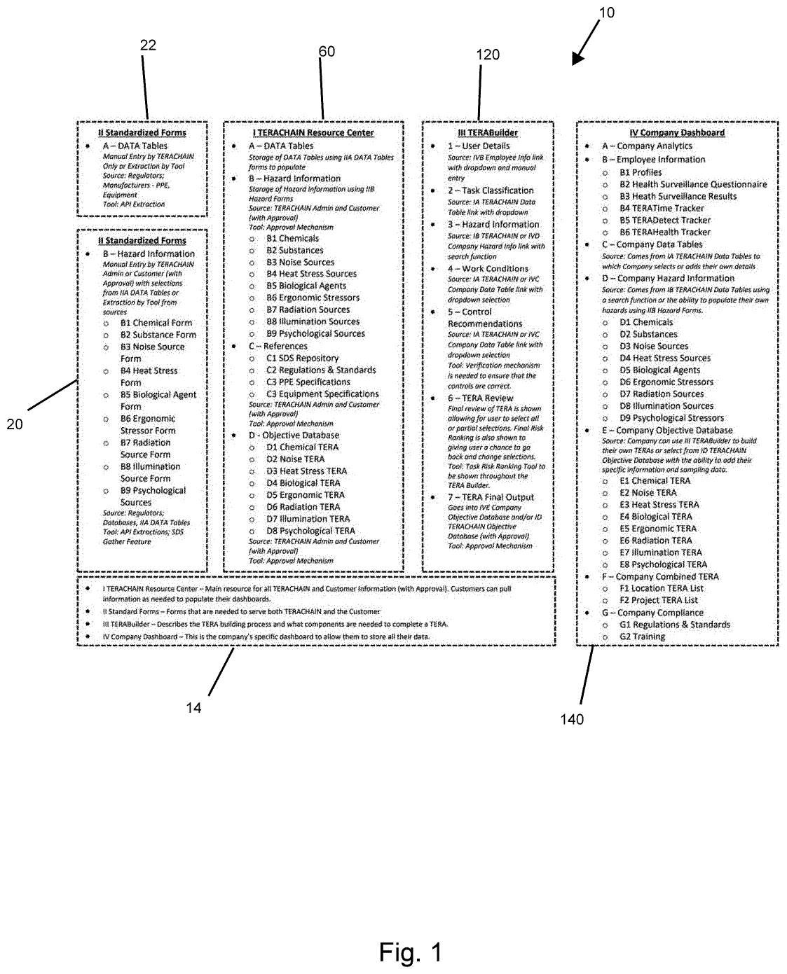

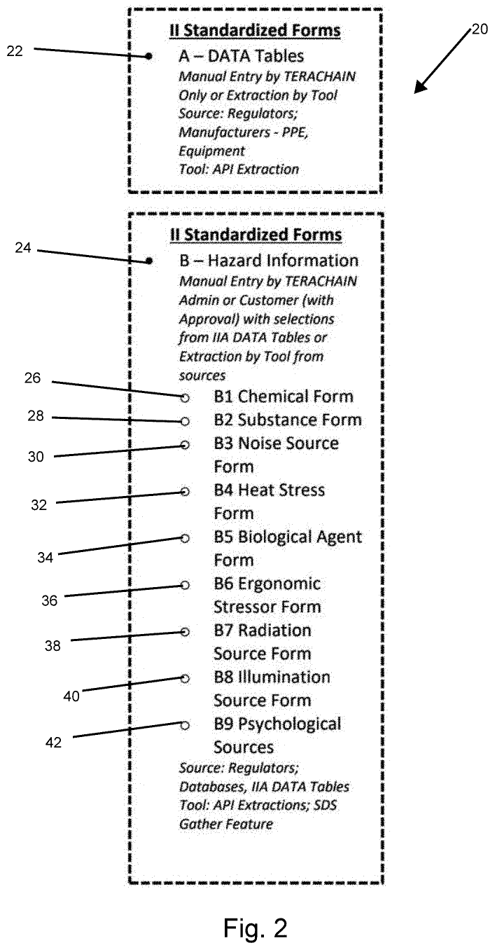

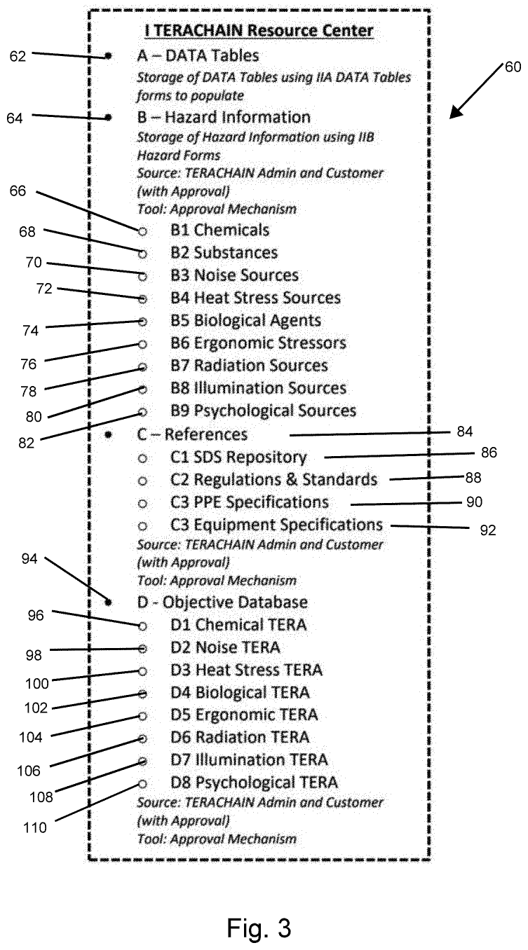

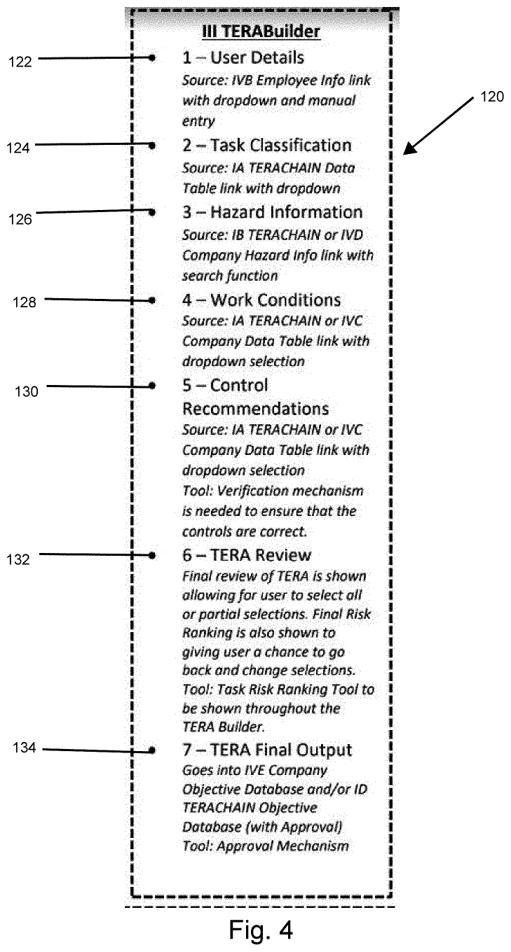

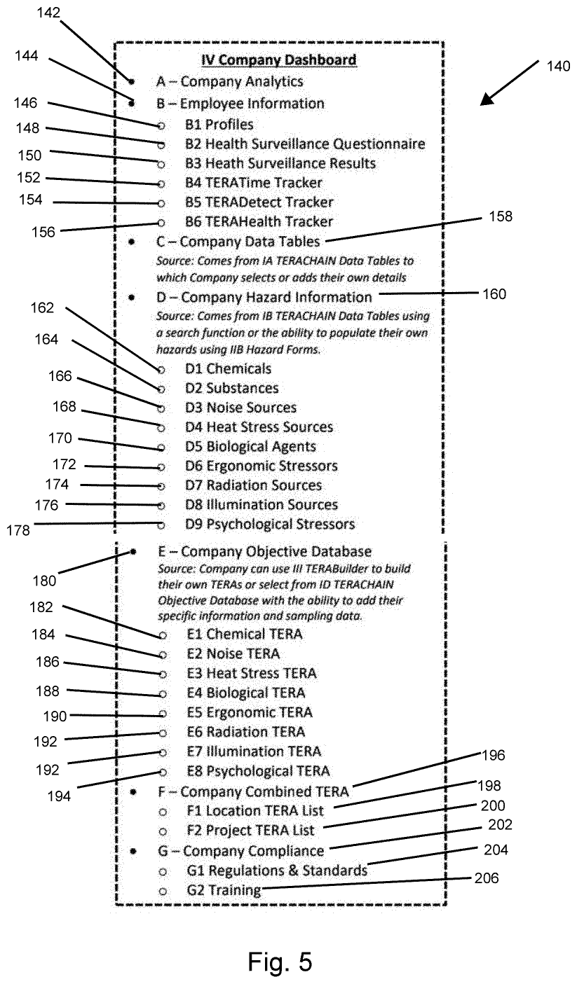

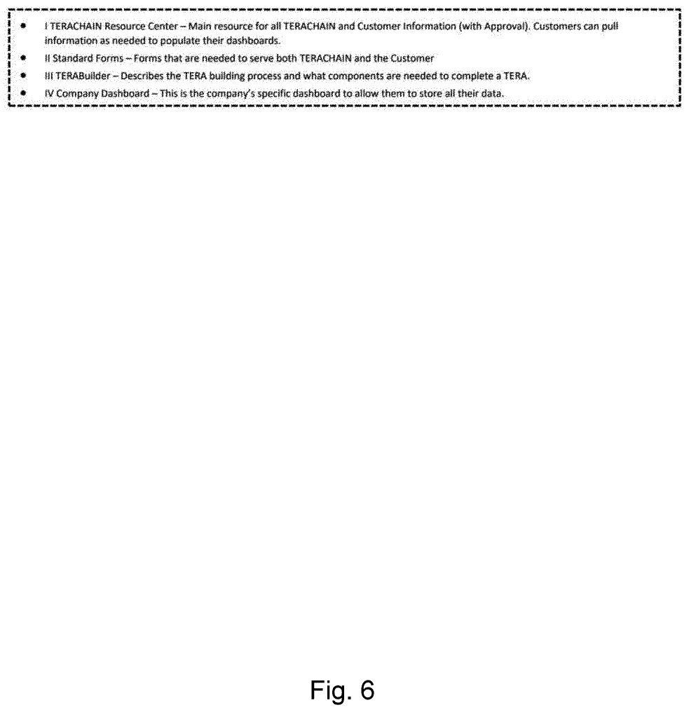

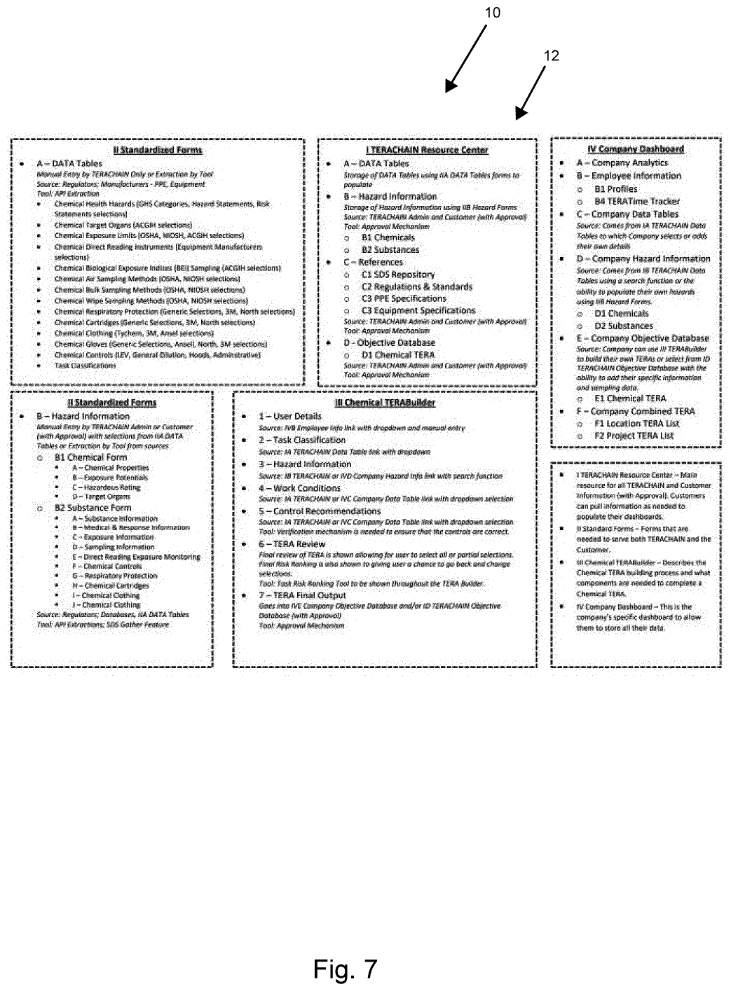

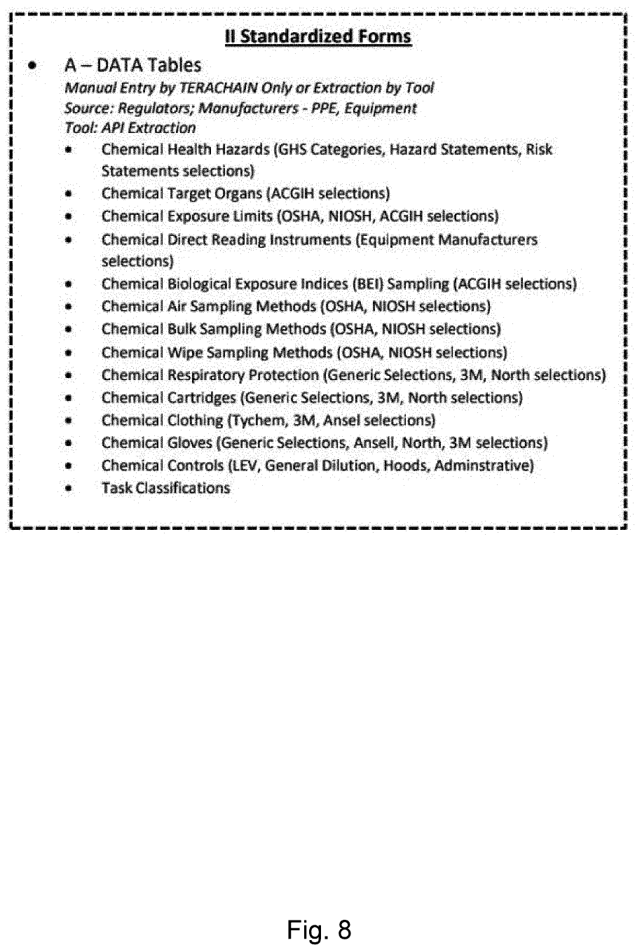

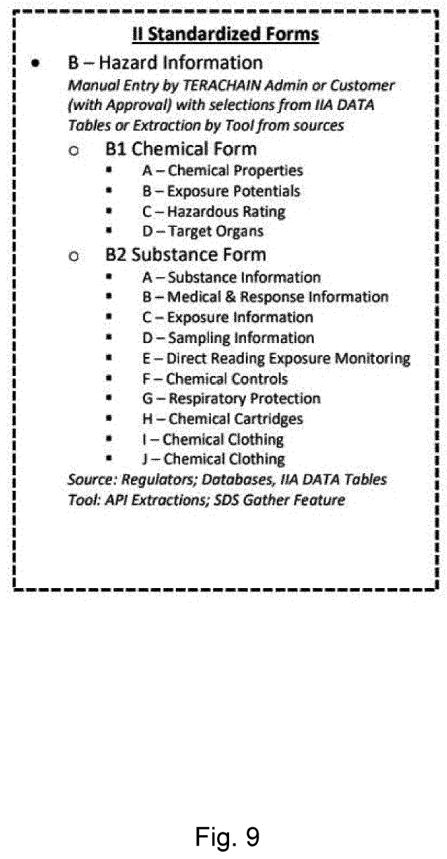

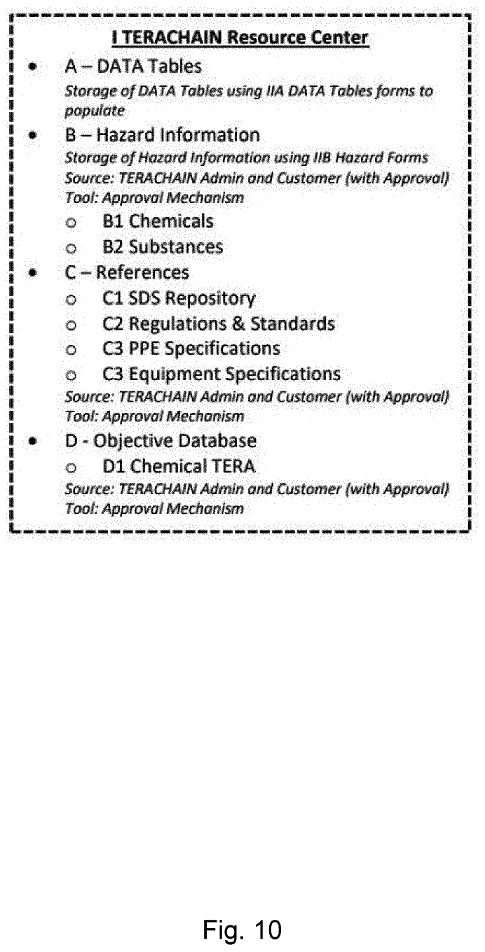

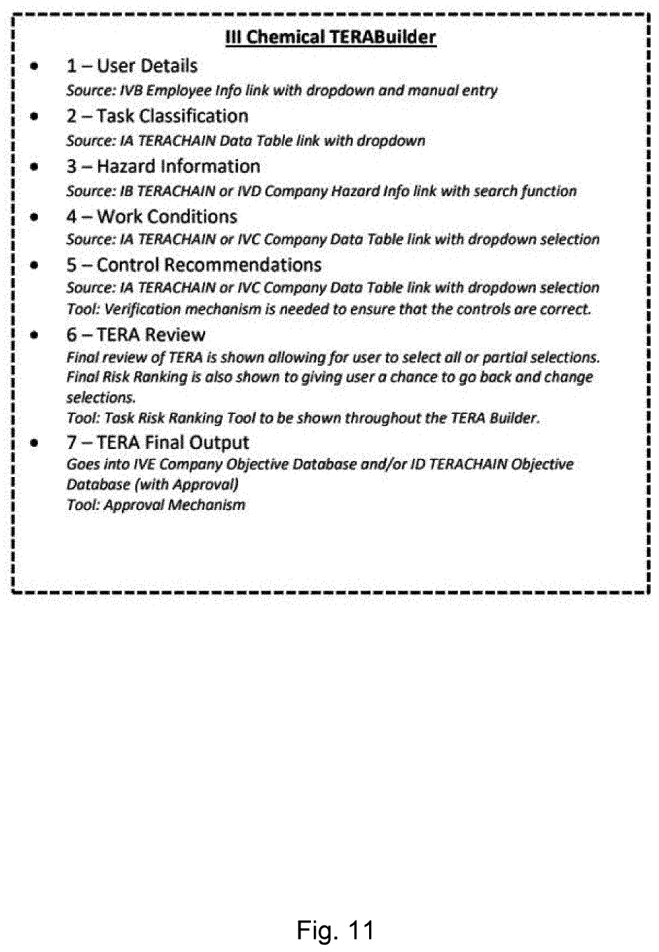

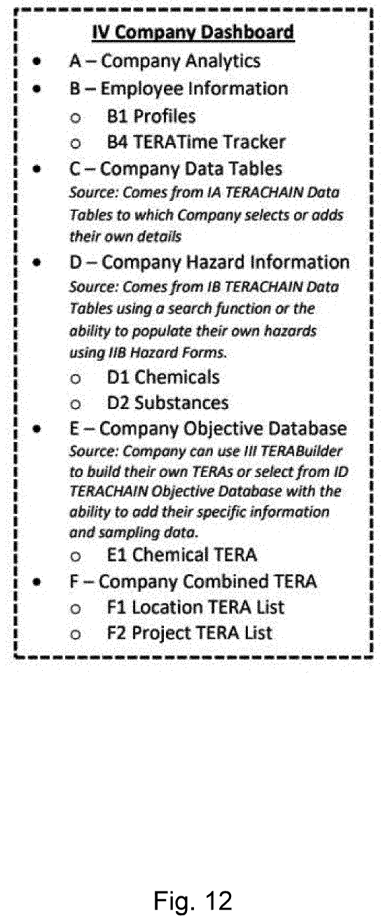

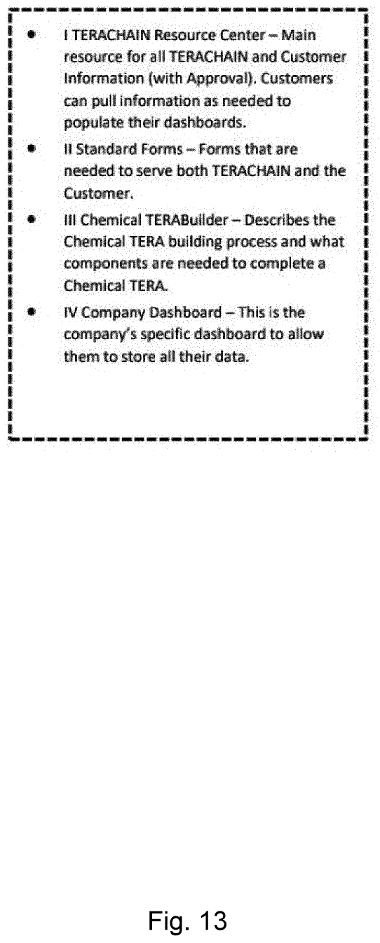

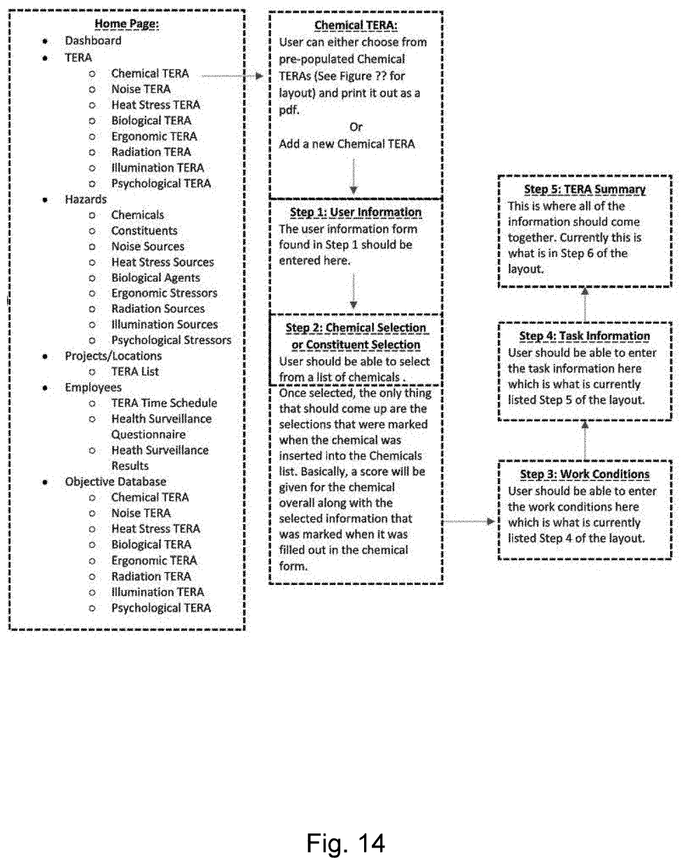

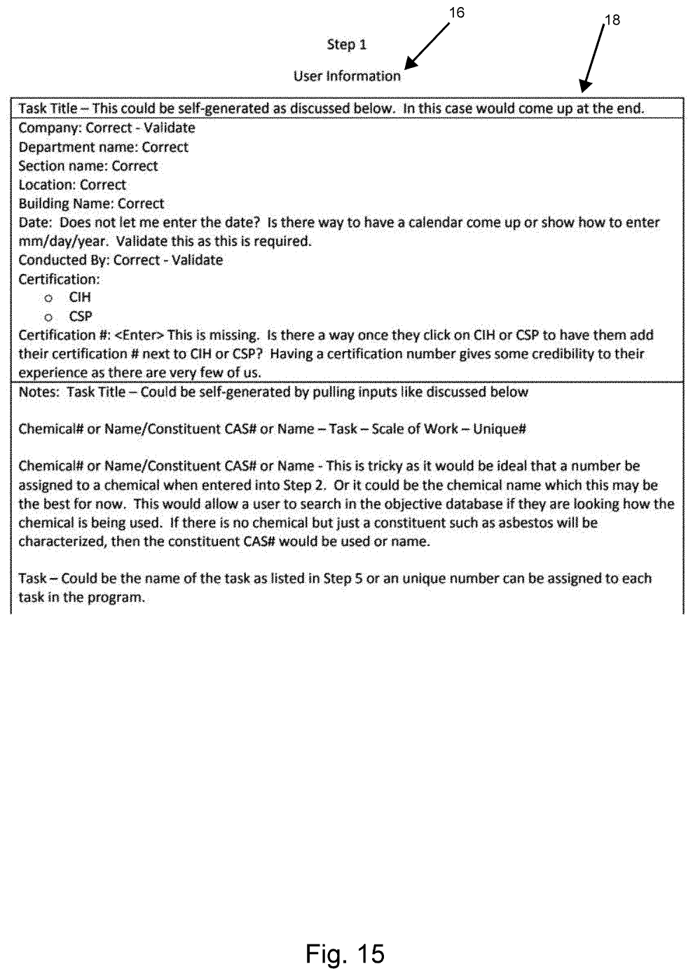

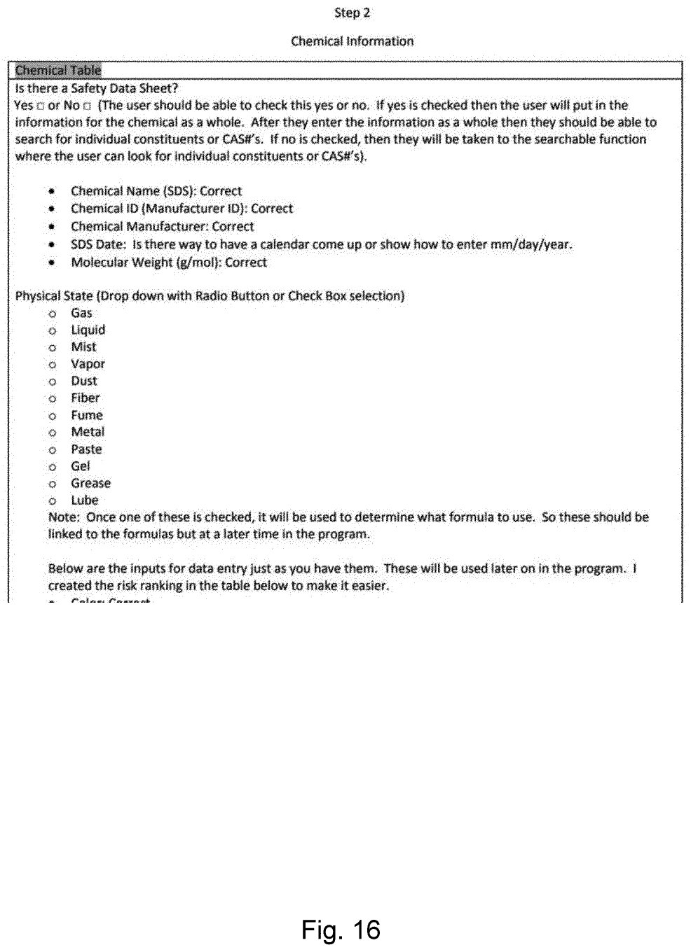

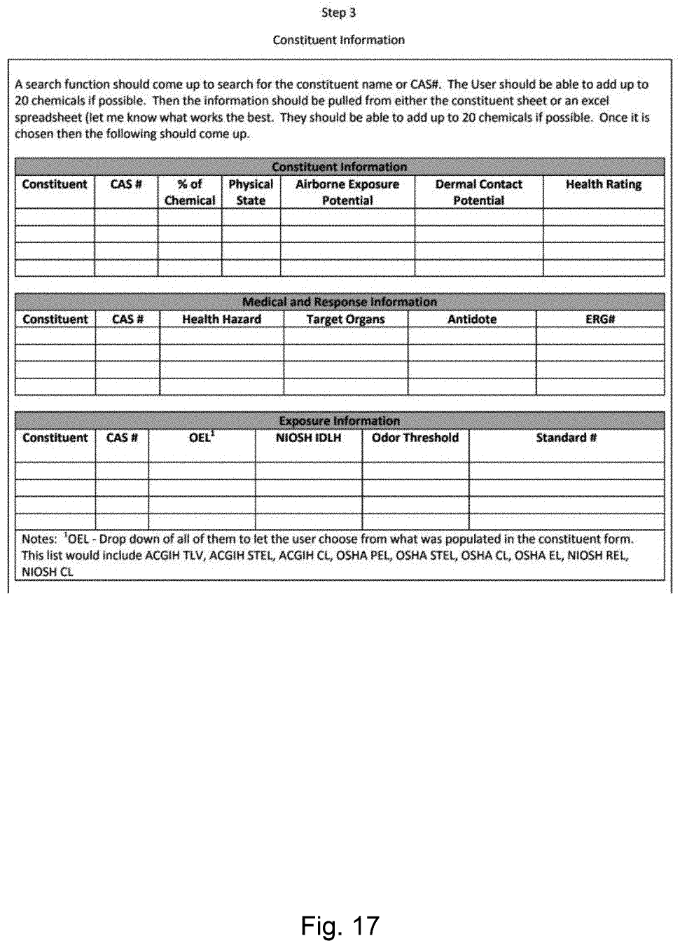

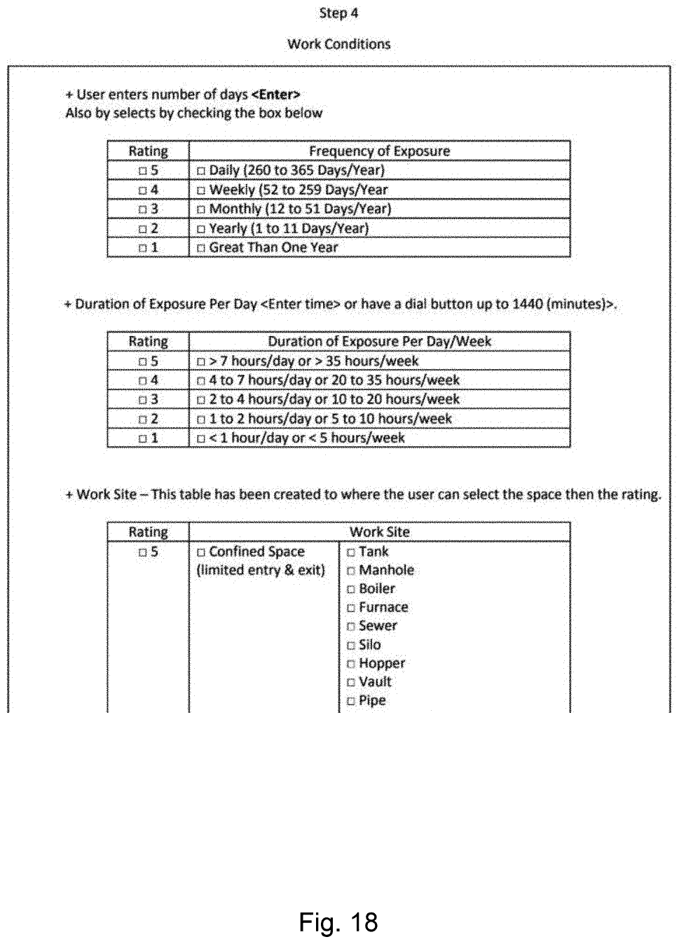

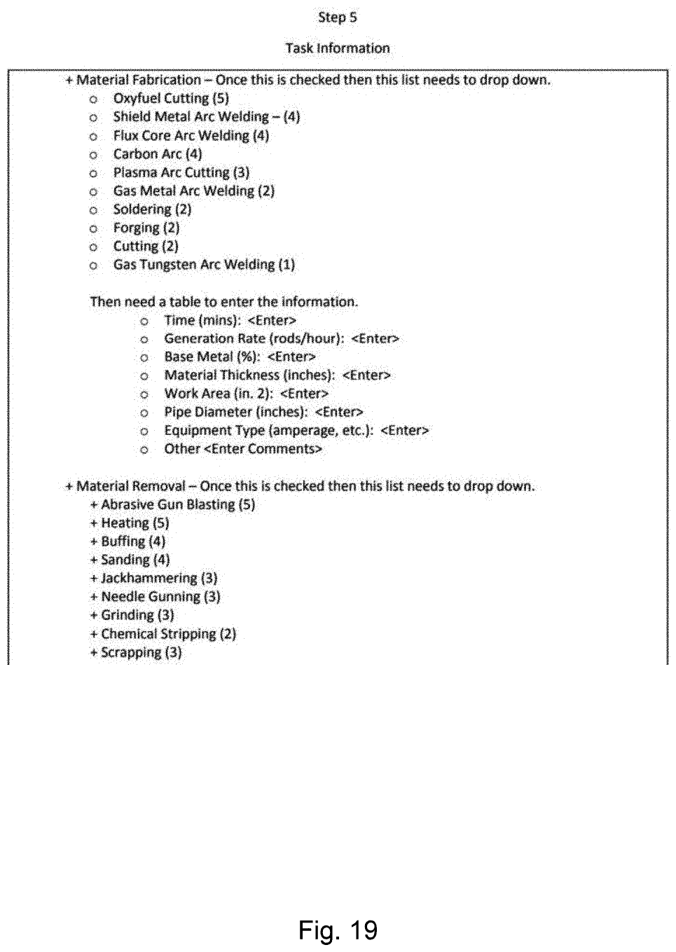

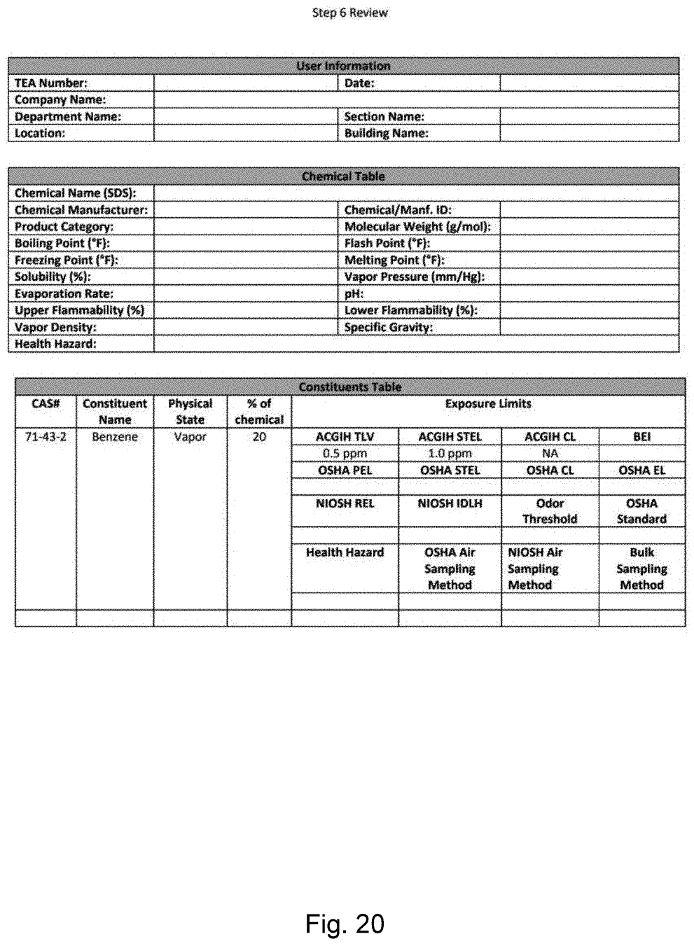

The following descriptions are in reference to FIG. 1 through FIG. 20. The present invention is a system and method to characterize risk of occupational health hazards and implement control measures to protect workers, with real time evaluations of environmental conditions and exposure concentrations. Throughout the specification, a “user” may be a person or any entity who wishes to evaluate a task exposure risk assessment and/or use the system and perform the methods and processes described herein; a user (or “individual”, or “entity”, or “company”) may be any entity, professional, individual, or worker evaluating environmental health and safety and/or providing or processing improvements to the health and safety of an environment.

The system and method to characterize risk of occupational health hazards and implement control measures to protect workers, with real time evaluations of environmental conditions and exposure concentrations comprises a customer management engine, a healthy engine, an environment engine, a user engine, a management engine, a graphical user interface (“GUI”) engine, a search engine, an results engine, a report management engine, a communication engine, and a database.

Additionally, the present invention is available in the form of a mobile application which can operate on a mobile device (such as a smartphone, a tablet, etc.) that possesses computing capability and network connectivity. It should be noted that entities and users may use an integrated application; however, separate applications, which are specifically designed for the entities and individual users respectively, may be provided. As used herein, the term “engine” is used to refer to collections of programs which are grouped based upon function.

The graphical user interface (or “GUI”) engine generates graphical data for displaying a user interface which enables users and/or entities to interact with the present invention. A user can input information into the user interface and the graphical user interface engine relays the information to other engines for further processing. For example, the user interface may include a login box into which users can enter login credentials, and then the credentials are transmitted from the graphical user interface engine to the worker management engine for user authentication. If the login credentials do not match with those in the database, the graphical user interface engine will generate a pop-up window requiring the customer to re-enter the credentials. In a preferred embodiment, the graphical user interface engine generates a graphical user interface showing common environmental conditions, common safety hazards, and common solutions on various pages.

In one embodiment, a user enters information related to environmental conditions through the graphical user interface. The environmental information is stored in the memory of the environmental processing engine. Additionally, the user may enter individual information related to the user and/or worker. The worker information will be stored in a memory and processor by the worker engine. The information stored and processed can then be uploaded to the task exposure risk assessment builder. The task exposure risk assessment builder provides a number of functions, such as but not limited to, a standardization of task classifications, interpolation of hazard information, work condition assessments, provide various control recommendations, provide a review of this information, and generate a plurality of reports. These features, other functionality, and components, and methods of use will become more clear in the specification herein.

Although the invention has been explained in relation to its preferred embodiment, it is to be understood that many other possible modifications and variations can be made without departing from the spirit and scope of the invention.

System

With reference to the figures, a system and method to characterize risk of occupational health hazards and implement control measures to protect workers, with real time evaluations of environmental conditions and exposure concentrations 10, and platform, and methods of use are presented (hereafter known as “task exposure risk assessment system” or “occupational health hazard system” or simply “system”). Task exposure risk assessment system 10 is formed of any suitable size, shape and design. In the arrangement shown, as one example, system 10, may comprise remote servers, databases, application servers, application databases, application programming interfaces, user databases, hazard databases, environmental databases, global positioning features, mobile applications, and/or computers that fulfill the functions disclosed herein, also includes, in the embodiment depicted, a graphical user interface 12, a legend 14, a plurality of users 16, and user accounts 18, among other components, features, and functions.

In the arrangement shown, as one example, system 10 may comprise remote servers, databases, and/or computers that fulfill the functions disclosed and described herein. In the embodiment depicted, occupational health hazard system 10 comprises an application server. Application server comprises one or more computer systems adapted to transmit and receive data regarding selected datasets related to various users and/or datasets related to multiple users and/or datasets related to environmental conditions and/or datasets related to hazards. Application server is adapted to query database with unique identification codes to retrieve a list of users and/or hazards and/or generate reports related to various environmental conditions. Application server is also adapted to query a standardized form database. Additionally, the application server may communicate with a mobile application, which is adapted to present the occupational health and safety information in a form conducive to being viewed on a mobile device and/or handheld device.

The appearance of the presentation of the user occupational health and safety information and/or the process windows for researching hundreds of occupational health situations and/or scenarios and/or occupational health events may be customized with data of particular relevance to the user and/or the product and/or the occupational health or safety event type or location. As one example, the appearance of windows and/or the process for researching hundreds of occupational health hazards may be customized for user information, entity information, hazard information, geographic information and/or other information related to occupational health and safety.

As one of ordinary skill in the art may understand, application server, product database, and other databases mentioned herein may be implemented in one or more servers. Additionally, multiple servers may have mirrored data to prevent data loss in case of disk failure and/or to decrease access and response times for database queries. In alternative embodiments, application server, and other database procedures may be carried out on computer-readable instructions and data stored on the customer's mobile computing device. Additionally, system 10 includes an application programming interface (“API”) which includes tools and resources enabling a user to operate the embodiments herein.

Graphical User Interface:

In the arrangement shown, as one example, system 10 includes a graphical user interface 12. Graphical user interface 12 is formed of any suitable size shape and design and is configured to present user and occupational health and safety data to a user in an easy to use manner. Additionally, graphical user interface 12 is configured to generate reports related to occupational health and safety data which provide results and/or suggestions for the mitigation of potential occupational health and safety hazards. Said another way, graphical user interface 12 is configured to allow users to set up online scenarios of real world and/or potential real world situations in which a user can identify, measure, analyze, compute, and make changes for a plurality of occupational health and safety scenarios. Additionally, graphical user interface 12 is configured to provide a plurality of users with access to these services in an easy to use way such that a user need not be a highly qualified industrial hygienist to perform the identification and analysis, or any other steps of the process (as disclosed further herein).

Furthermore, graphical user interface 12 provides an easy way for a user, experienced or inexperienced, to upload information to a database, enter information into a database, storing that information, sharing that information, interpolating this information, and using that information to provide resolutions and/or measurable standardized easy to interpret reports, among other services, as well as mitigation options. Graphical user interface 12 is an exemplary method by which systems of the present disclosure may represent, provide, connect, book, retrieve, and organize, and report, and more.

In the arrangement shown, as one example, graphical user interface 12 includes a standardization page 20, a resource center page 60, a task exposure risk assessment builder page 120, and a dashboard 140, among other pages, features, and other components.

Standardization Page:

In the arrangement shown, as one example, graphical user interface includes standardized forms 20 (or “standard forms page”, or “standardization page”). Standardization page 20 is formed of any suitable size, shape, and design and is configured to provide a standard data entry set of forms and processes in which information can be entered manually through the TeraChain system and/or manually through the system 10. Additionally, the system provides the standard entry by tool source, manufacturer, regulators, and users entry. In the arrangement shown, as one example, application programming interface extraction is available along with these other features which provide standardization and standard forms for the entire process of the methods disclosed herein.

In the arrangement shown, as one example, standardization page 20 includes a data tables feature 22 (or “table feature”). Data table feature 22 is formed of any suitable size shape and design and includes but is not limited to a tab on the standardization page 20 for accessing the tables feature 22. In the arrangement shown, as one example, the tables feature 22 is configured to provide preset tables for entering data or retrieving data. A number of tables have been created and provided with the system to provide for standardized tables. Additionally, the tables feature 22 provides for uploading or entering customized information into the tables feature 22. This includes the modification of almost any element within the tables. Additionally, system 10 provides for machine learning which can modify the tables according to specific rules and circumstances and learned rules and circumstances.

In the arrangement shown, as one example, standardization page 20 includes a hazard information feature 24. Hazard information feature 24 is formed of any suitable size, shape, and design and includes but is not limited to a tab on the standardization page 20 for accessing the hazard information feature 24 and providing for standardization. In the arrangement shown, as one example, the hazard information feature 24 is configured to provide a user with the ability to search existing hazards in the system, to search for hazards uploaded and entered into the system by others, as well as the ability to customize or enter in new hazards. This includes the modifications of almost any element within the hazard information.

In the arrangement shown, as one example, the hazard information feature 24 includes a chemicals form feature 26, a substances form feature 28, a noise forms feature 30, a heat stress forms feature 32, a biological agent forms feature 34, an ergonomic stressor forms feature 36, a radiation sources forms feature 38, an illumination sources forms feature 40, a psychological forms feature 42, a source feature, a regulator feature, a database, a data tables tool feature, and an extraction tool, among other options, controls, features, and components.

Resource Center Page:

In the arrangement shown, as one example, graphical user interface includes a resource center 60 (or “resource center page”, or “resource center feature”). Resource center page 60 is formed of any suitable size, shape, and design and is configured to provide a storage of the various data uploaded to system 10 along with manual and/or automated retrieval based on a predetermined set of rules.

Additionally, resource center 60 and resource center page 60 provides sets of forms and processes in which information can be entered manually through the TeraChain system and/or manually through the system 10, and subsequently uploaded and saved. Additionally, the system provides the standard entry by tool source, manufacturer, regulators, and users entry. In the arrangement shown, as one example, application programming interface extraction is available along with these other features which provide standardization and standard forms for the entire process of the methods disclosed herein.

In the arrangement shown, as one example, resource center page 60 includes a data tables feature 62 (or “table feature”). Data table feature 62 is formed of any suitable size shape and design and includes but is not limited to a tab on the resource center page 60 for accessing the tables feature 62. In the arrangement shown, as one example, the tables feature 62 is configured to provide preset tables for entering data or retrieving data. A number of tables have been created and provided with the system to provide for standardized tables. Additionally, the tables feature 62 provides for uploading or entering customized information into the tables feature 62. This includes the modification of almost any element within the tables. Additionally, system 10 provides for machine learning which can modify the tables according to specific rules and circumstances and learned rules and circumstances. The resource center page 60 also providence for viewing this information, for flexibility in use of the standardized presentation of these resources.

In the arrangement shown, as one example, resource center page 60 includes a hazard information feature 64. Hazard information feature 64 is formed of any suitable size, shape, and design and includes but is not limited to a tab on the resource center page 60 for accessing the hazard information feature 64 and providing for standardization. In the arrangement shown, as one example, the hazard information feature 64 is configured to provide a user with the ability to search existing hazards in the system, to search for hazards uploaded and entered into the system by others, as well as the ability to customize or enter in new hazards. This includes the modifications of almost any element within the hazard information.

In the arrangement shown, as one example, the hazard information feature 64 includes a chemicals feature 66, a substances feature 68, a noise feature 70, a heat stress feature 72, a biological agents feature 74, an ergonomic stressor feature 76, a radiation feature 78, an illumination sources feature 80, and a psychological feature 82, among other options, controls, features, and components.

In the arrangement shown, as one example, resource center page 60 includes a plurality of references 84. Plurality of references 84 may be any relevant references which are uploaded and predetermined to be applicable for system 10. In one embodiment, these references 84 includes, but are not limited to, safety data sheets 86, regulations and standards 88, personal protective equipment specifications 90, equipment specifications 92, approval mechanisms 94, a combination thereof, and the like, among other reference materials, and approval mechanisms and tools.

In the arrangement shown, as one example, the resource center page 60 includes an objective database feature 96. Objective database feature 96 of the resource center page 60 is formed of any suitable size, shape, and design and includes but is not limited to a tab on the resource center page 60 for accessing the objective database feature 96. In the arrangement shown, as one example, the objective database feature 96 is configured to provide standard forms for providing objective evaluations and reports of task exposure risk assessments. Additionally, and said another way, the objective database feature 96 provides searchable identification for various task exposure risk assessments, and the ability to add customized information to task exposure risk assessments, and the ability to sample data associated with task exposure risk assessments. This includes the modifications of almost any element within the objective database feature.

In the arrangement shown, as one example, the objective database feature 96 includes a chemical task exposure risk assessment feature 98, a noise task exposure risk assessment feature 100, a heat task exposure risk assessment feature 102, a biological task exposure risk assessment feature 104, an ergonomic task exposure risk assessment feature 106, a radiation task exposure risk assessment feature 108, an illumination task exposure risk assessment feature 110, a psychological task exposure risk assessment feature 112, and an approval mechanism, among other options, controls, features, and components.

Task Exposure Risk Assessment Builder Page:

In the arrangement shown, as one example, graphical user interface includes a task exposure risk assessment builder 120 (or “task exposure risk assessment builder feature”, or “task exposure risk assessment builder page”, or simply “TERA Builder Page”). Task exposure risk assessment builder page 120 is formed of any suitable size, shape, and design and is configured to provide a system and method of use for an industrial hygienist to quickly and efficiently evaluate exposure risk assessments in real time as well as provide near instant mediation recommendations for controlling and mitigating exposures. Said another way, the TERA builder 120 is configured to provide a means for identifying the task exposure risk, analyzing the task exposure risk for a plurality of workers, provide calculations and interpretations of this information based on a predetermined set of rules, and provide assessments and generate reports and provide recommendations for improved occupational health and safety in an environment.

In the arrangement shown, as one example, task exposure risk assessment builder 120 includes user details 122, a plurality of dropdown menus 124 which retrieves data from a plurality of information sources and/or databases, a plurality of task classifications 126, hazard information 128, work conditions 130, control recommendations 132, a task exposure risk assessment review 134, and a task exposure risk assessment final output 136, among other options, controls, features, and components.

Dashboard:

In the arrangement shown, as one example, task exposure risk assessment system 10 includes an initial starting page for launching, accessing, and viewing many of the different functionalities available in system 10, and additionally, launching the means to add information and create scenarios not previously registered in the database. Alternatively, for devices and/or users not recognized in the database, the user will be redirected to a login page as the landing page (or “splash page”). The dashboard (or “dashboard page”) 140, for unregistered and/or unrecognized devices is a sign up and/or create account 18 (account further discussed herein) series of pages which guides a user and/or new user through a series of screens to guide the user in creating an account.

In this way, dashboard page 140 is formed of any suitable size, shape, and design and is configured to generally be the main page a user utilizes to start the process, and/or website and/or graphical user interface 12. In addition, and in the arrangement shown, the dashboard page 140 may also serve as a splash, oftentimes being the first page or a page by which a user lands after logging into their account 18. Additionally, the dashboard page 140 may provide a plurality of page links and/or tabs for navigating the graphical user interface 12.

Additionally, and said another way, the dashboard page 140 is configured to help a user manage accounts or access various accounts. In other words, the dashboard page 140 is configured to help a user choose an account or switch between different accounts as well as disable or activate various accounts. Additionally, this feature is designed to supply other actions and components, such as deleting accounts and/or launching previously disabled accounts. Additionally, in the dashboard page 140, a user may select the desired account/product and focus on a particular scenario and/or client which the user is aiding.

In this arrangement, as one example, a user accesses their account via a login page as part of the graphical user interface 12. In this way, the login page is the entrance page to the graphical user interface 12. In this way, the login page serves the purpose of security and privacy and customization, allowing for the customization of a dashboard and favorites feature and the like, and is the page through which a user gains access to system 10 by identifying and authenticating themselves. In the arrangement shown, as one example, the login page includes a display, a username, a password, a log in, among other credentials, features, and components, and may also include bio-identification.

In the arrangement shown, as one example, the dashboard page 140 includes an analytics tab 142—including an analytics feature and analytics components—a worker information tab 144, a tables tab 158, a hazard information tab 160, an objective database 180, a combined task exposure risk assessment 198, and a compliance feature 204, among other options, controls, features, and components.

Worker Information Feature: In the arrangement shown, as one example, the dashboard page 140 includes an employee information feature 144 (or “worker information” feature). Worker information feature 140 is formed of any suitable size, shape, and design and includes but is not limited to a tab on the dashboard page 140 for accessing the worker information feature 144. In the arrangement shown, as one example, the worker information feature 144 is configured to provide uploading and entering information related to a worker and/or a plurality of workers. This information may include, but is not limited to, demographic information related to a worker.

In the arrangement shown, as one example, the worker information tab 144 includes a worker profile 146, a health questionnaire 148, health results 150, a task exposure risk assessment time of exposure 152, a task exposure risk assessment detection feature 154, and a task exposure risk assessment health feature 156, among other options, controls, features, and components.

Tables Feature: In the arrangement shown, as one example, the dashboard page 140 includes a tables feature 158 (or “table feature”). Tables feature 158 is formed of any suitable size shape and design and includes but is not limited to a tab on the dashboard page 140 for accessing the tables feature 158. In the arrangement shown, as one example, the tables feature 158 is configured to provide preset tables for entering data or retrieving data. A number of tables have been created and provided with the system to provide for standardized tables. Additionally, the tables feature 158 provides for uploading or entering customized information into the tables feature 158. This includes the modification of almost any element within the tables. Additionally, system 10 provides for machine learning which can modify the tables according to specific rules and circumstances and learned rules and circumstances.

Hazard Information Feature: In the arrangement shown, as one example, the dashboard page 140 includes a hazard information feature 160. Hazard information feature 160 is formed of any suitable size, shape, and design and includes but is not limited to a tab on the dashboard page 140 for accessing the hazard information feature 160. In the arrangement shown, as one example, the hazard information feature 160 is configured to provide a user with the ability to search existing hazards in the system, to search for hazards uploaded and entered into the system by others, as well as the ability to customize or enter in new hazards. This includes the modifications of almost any element within the hazard information.

In the arrangement shown, as one example, the hazard information feature 160 includes a chemicals sources feature 162, a substances sources feature 164, a noise sources feature 166, a heat sources feature 168, a biological sources feature 170, an ergonomic sources feature 172, a radiation sources feature 174, an illumination sources feature 176, and a psychological sources feature 178, among other options, controls, features, and components.

Objective Database Feature: In the arrangement shown, as one example, the dashboard page 140 includes an objective database feature 180. Objective database feature 180 is formed of any suitable size, shape, and design and includes but is not limited to a tab on the dashboard page 140 for accessing the objective database feature 180. In the arrangement shown, as one example, the objective database feature 180 is configured to provide standard forms for providing objective evaluations and reports of task exposure risk assessments. Additionally, and said another way, the objective database feature 180 provides searchable identification for various task exposure risk assessments, and the ability to add customized information to task exposure risk assessments, and the ability to sample data associated with task exposure risk assessments. This includes the modifications of almost any element within the objective database feature.

In the arrangement shown, as one example, the hazard information feature 160 includes a chemical task exposure risk assessment feature 162, a noise task exposure risk assessment feature 184, a heat task exposure risk assessment feature 186, a biological task exposure risk assessment feature 188, an ergonomic task exposure risk assessment feature 190, a radiation task exposure risk assessment feature 192, an illumination task exposure risk assessment feature 194, and a psychological task exposure risk assessment feature 196, among other options, controls, features, and components.

Combined Task Exposure Risk Assessment Feature:

In the arrangement shown, as one example, the dashboard page 140 includes a combined task exposure risk assessment feature 198. Combined task exposure risk assessment feature 198 is formed of any suitable size, shape, and design and includes but is not limited to a tab on the dashboard page 140 for accessing the task exposure risk assessment feature 198. In the arrangement shown, as one example, the combined task exposure risk assessment feature 198 is configured to provide a combination of task exposure risk assessments including, but not limited to, the evaluation, and analysis of a task exposure risk assessment including location 200, user 14, and project 202 information.

Compliance Feature:

In the arrangement shown, as one example, the dashboard page 140 includes a compliance feature 204. Compliance feature 204 is formed of any suitable size, shape, and design and includes but is not limited to a tab on the dashboard page 140 for accessing the compliance feature 204. In the arrangement shown, as one example, the compliance feature 204 is configured, not only to provide standardization, but to provide a report and checklist for compliance in a plurality of industries which includes the integration of regulations 206, standards 208, training compliance 210, and more. Additionally, and in the arrangement disclosed herein, the compliance feature provides alerts if compliance is not met for any particular scenario or set of circumstances.

In Operation/Method of Use:

As one example, a system and method to characterize risk of occupational health hazards and implement control measures to protect workers, with real time evaluations of environmental conditions and exposure concentrations. Said another way, the present disclosure provides a system and method for characterizing the risk of various occupational health hazards across various industries, implementing and providing control measures that can be adopted to protect workers in such hazards or situations, and even provide real-time evaluations and recommendations based on existing conditions of environmental conditions to mitigate exposure concentrations of various hazards that may be experienced by or induced upon a worker in an environment.

As one example, a user uploads environmental and worker information to evaluate risk exposure of a particular situation. As another example, a user retrieves a pre-loaded set of data and rules associated with a known environment or generally known environment. Additionally, in this circumstances known user information can be retrieved or user data can be uploaded. The system then builds a task exposure risk assessment based on the data entered or data selected. The system then provides generated reports so a user can change the environment or make other changes. Additionally, in another example, the system may provide alerts of conditions which do not meet local regulations or standard requirements for a particular jurisdiction or industry. As another example, the system may generate real-time recommendations of how to improve the circumstances, and/or make real-time recommendations associated with an uploaded sample, whether it be an air sample, a water sample, a combination thereof, or the like.

It will be appreciated by those skilled in the art that other various modifications could be made to the device without parting from the spirit and scope of this disclosure. All such modifications and changes fall within the scope of the claims and are intended to be covered thereby.

What is claimed:

1. A system for characterizing risk of occupational health hazards, the system comprising:
a computer system;
the computer system comprising a database, the computer system configured to maintain a user profile in the database;
the computer system configured to maintain a plurality of data tables;
the computer system configured to maintain a set of hazard information;
a graphical user interface;
the graphical user interface having a standard forms page;
the graphical user interface having a resource center page;
the graphical user interface having a dashboard page;
a task exposure risk assessment builder;
the task exposure risk assessment builder having a processor for integrating;
wherein each user profile comprises at least one characteristic of a user;
wherein each user profile accepts a plurality of inputs, in the form of a plurality of characteristics, toward creating the user profile;
wherein the computer system is configured to store data corresponding to the defined environment;
wherein the computer system is configured to store data corresponding to a user profile;
wherein the user profile accesses a graphical user interface to upload a set of information;
wherein the computer system stores the set of information associated with the defined environment.

2. The system of claim 1, further comprising: wherein the user profile is at least one person who performs a task exposure risk assessment.

3. The system of claim 1, further comprising: a geographic location; the geographic location having a defined space.

4. The system of claim 1, further comprising: a plurality of hazard types.

5. The system of claim 1, further comprising: wherein the computer system is configured to define a standardized document page, wherein the standardized document page displays a plurality of documents associated with the information related to the set of hazard information.

6. The system of claim 1, further comprising: a plurality of hazard information, wherein the computer system is further configured to define a plurality of characteristics that are to be displayed in the graphical user interface in response to a user input.

7. A system to characterize risk of occupational health hazards and implement control measures to protect workers, with real time evaluations of environmental conditions and exposure concentrations, the system comprising:
a computer system;
a standardized forms system;
a resource center;
a dashboard;
a defined space;
a plurality of hazards;
each of the pluarlity of hazards having a data set;
wherein the data set of the each of the plurality of hazards defines parameters associated with each of the plurality of hazards;
wherein the parameters of the each of the plurality of hazards is used to determine an output based on a predetermined set of rules.

8. The system of claim 7, further comprising:
a graphical user interface;
the graphical user interface having a standardized forms page;
the graphical user interface having a resource center page;
the graphical user interface having a dashboard page;

9. The system of claim 7, further comprising: a predetermined set of rules for generating an output based on an input of the parameters associated with each of the plurality of hazards.

10. The system of claim 7, further comprising:
a plurality of worker profiles;
the plurality of worker profiles each having a set of parameters;
a predetermined set of rules for generating an output based on an input of the parameters associated with each of the plurality of worker profiles.

11. The system of claim 7, further comprising:
a plurality of regulations;
the plurality of regulations associated with a set of rules;
a plurality of standards;
wherein the plurality of regulations is integrated into the computer system.

12. The system of claim 7, further comprising:
an objective database;
the objective database having a chemical task exposure risk assessment;
the objective database having a noise task exposure risk assessment;
the objective database having a heat task exposure risk assessment;
the objective database having a biological task exposure risk assessment;
the objective database having a radiation task exposure risk assessment;
the objective database having an ergonomic task exposure risk assessment;
the objective database having an illumination task exposure risk assessment;
the objective database having a psychological task exposure risk assessment;
an approval mechanism;
wherein the approval mechanism evaluates a risk exposure associated with each parameter of the objective database.

13. The system of claim 7, further comprising:
a compliance check system;
wherein the compliance check system sends an alert to a user when a parameter of the compliance check system is outside a defined range.

14. The system of claim 7, further comprising:
a combined task exposure risk assessment;
wherein the combined task exposure risk assessment combines a plurality of parameters from a plurality of task exposure risk assessments based on a predetermined set of rules to generate a report;
wherein the report indicates a set of risks based on a combination of a plurality of hazards.

15. The system of claim 7, further comprising:
at least one reference.

16. A method to characterize risk of occupational health hazards and implement control measures to protect workers, with real time evaluations of environmental conditions and exposure concentrations, the steps comprising:
providing a computer system;
providing a set of standardized forms;
providing a resource center;
providing a dashboard;
defining a space;
identifying at least one hazard within the space;
analyzing at least one hazard within the space based on a predetermined set of rules.

17. The method of claim 16, further comprising the steps:
maintaining a user profile;
uploading a set of parameters associated with at least one hazard;
evaluating the set of parameters associated with at least one hazard.

18. The method of claim 16, further comprising the steps:
storing data corresponding to a set of parameters associated with at least one hazard.

19. The method of claim 16, further comprising the steps:
searching a database by at least one hazard, subsequently retrieving a plurality of parameters associated with the at least one hazard.

20. The method of claim 16, further comprising the steps:
uploading a set of parameters associated with at least one worker;
storing data corresponding to the set of parameters associated with at least one worker;
evaluating the set of parameters associated with at least one worker.
